(19) United States
(12) Patent Application Publication
(10) Pub. No.: US 2026/0269072 A1
(43) Pub. Date: Sep. 10, 2026

(54) ARTIFICAL INTELLIGENCE ASSISTED PSYCHOPATHOLOGY EVALUATION SYSTEM

(71) Applicant: MedLink Global, Inc., San Ramon, CA (US)

(72) Inventors: Mark A BLAIS, Melrose, MA (US); Ran YANG-CHAWLA, San Ramon, CA (US)

(21) Appl. No.: 19/445,513

(22) Filed: Jan. 10, 2026

Related U.S. Application Data
(60) Provisional application No. 63/744,175, filed on Jan. 11, 2025.

(51) Int. Cl. G16H 50/30

(52) U.S. Cl. CPC G16H 50/30

(57) ABSTRACT

The present disclosure provides methods and apparatuses for psychological assessment. An exemplary method includes: generating a script based on a generative model and a patient profile, wherein the generative model employs a structured psychological inventory comprising a plurality of items configured to measure symptoms and traits at multiple hierarchical levels; receiving responses to the script and storing the responses in a memory; computing a global general psychopathology factor based on the responses; aggregating the responses into a plurality of transdiagnostic dimensions; calculating a plurality of marker scores corresponding to clinically recognizable symptom clusters associated with each transdiagnostic dimension; determining one or more supplemental scales based on the responses; and generating a multi-level mental health profile for the user.

### CROSS-REFERENCE TO RELATED APPLICATIONS

The disclosure claims the benefits of priority to U.S. Provisional Application No. 63/744,175, filed on Jan. 11, 2025, which is incorporated herein by reference in its entirety.

### TECHNICAL FIELD

The present disclosure generally relates to the technical field of psychological assessment and, more particularly, to systems and methods for providing artificial intelligence assisted psychological assessment.

### BACKGROUND

Current clinical standards for assessing neuropsychiatric disorders primarily rely on structured questionnaires administered through verbal interaction between the subject and an evaluator, such as a clinician or caregiver. These assessments aim to quantify symptom severity and identify behavioral or psychological abnormalities.

Exemplary instruments include, for example: Hamilton Depression Rating Scale (HAM-D)—a 21-item questionnaire designed to evaluate depressive symptoms; Eating Attitudes Test (EAT-26)—a 26-item screening tool for identifying eating disorders; and Neuropsychiatric Inventory (NPI)—used to assess behavioral disturbances in patients with dementia.

While these instruments are widely accepted, they present two major challenges. First, these assessments require significant time and effort from healthcare professionals to administer, interpret, and document responses, especially in cases involving multiple sessions or complex symptom profiles. Second, the accuracy of these evaluations depends heavily on the patient's ability and willingness to provide truthful and detailed responses. Patients may consciously or unconsciously minimize or exaggerate symptoms, and those with cognitive impairments (e.g., Alzheimer's disease) may be unable to articulate their experiences effectively. This subjectivity can lead to inconsistent or unreliable psychopathology evaluation results.

The present disclosure provides methods and apparatuses for improving the way of providing psychological assessment.

### SUMMARY

In some embodiments, an exemplary computer-implemented method for psychological assessment is provided. The method includes: generating a script based on a generative model and a patient profile, wherein the generative model employs a structured psychological inventory comprising a plurality of items configured to measure symptoms and traits at multiple hierarchical levels; receiving responses to the script and storing the responses in a memory; computing a global general psychopathology factor based on the responses; aggregating the responses into a plurality of transdiagnostic dimensions; calculating a plurality of marker scores corresponding to clinically recognizable symptom clusters associated with each transdiagnostic dimension; determining one or more supplemental scales based on the responses; and generating a multi-level mental health profile for the user.

In some embodiments, an exemplary apparatus includes at least one memory for storing instructions and at least one processor. The at least one processor is configured to execute the instructions to cause the apparatus to perform: generating a script based on a generative model and a patient profile, wherein the generative model employs a structured psychological inventory comprising a plurality of items configured to measure symptoms and traits at multiple hierarchical levels; receiving responses to the script and storing the responses in a memory; computing a global general psychopathology factor based on the responses; aggregating the responses into a plurality of transdiagnostic dimensions; calculating a plurality of marker scores corresponding to clinically recognizable symptom clusters associated with each transdiagnostic dimension; determining one or more supplemental scales based on the responses; and generating a multi-level mental health profile for the user.

In some embodiments, an exemplary non-transitory computer readable storage medium stores a set of instructions. The set of instructions are executable by one or more processing devices to cause an apparatus to perform: generating a script based on a generative model and a patient profile, wherein the generative model employs a structured psychological inventory comprising a plurality of items configured to measure symptoms and traits at multiple hierarchical levels; receiving responses to the script and storing the responses in a memory; computing a global general psychopathology factor based on the responses; aggregating the responses into a plurality of transdiagnostic dimensions; calculating a plurality of marker scores corresponding to clinically recognizable symptom clusters associated with each transdiagnostic dimension; determining one or more supplemental scales based on the responses; and generating a multi-level mental health profile for the user.

### BRIEF DESCRIPTION OF THE DRAWINGS

The accompanying drawings, which are incorporated in and constitute a part of this specification, illustrate embodiments consistent with the present disclosure and, together with the description, serve to explain the principles of the present disclosure.

FIG. 1 illustrates a block diagram of an environment for performing psychological assessment using a generative Artificial Intelligence (AI) model, in accordance with an exemplary embodiment of the present disclosure.

FIG. 2 illustrates a block diagram of a server configured for performing psychological assessment for a communication network, in accordance with an exemplary embodiment of the present disclosure.

FIG. 3 shows a psychological assessment method designed to evaluate mental health across multiple hierarchical levels, in accordance with an exemplary embodiment of the present disclosure.

FIG. 4 shows a psychological assessment method designed to evaluate psychopathology across multiple hierarchical levels of specificity, in accordance with an exemplary embodiment of the present disclosure.

FIG. 5 is a schematic diagram illustrating a hierarchical structure of an assessment inventory, in accordance with an exemplary embodiment of the present disclosure.

FIG. 6 is a schematic diagram showing a system 600 for conducting a psychopathology evaluation interview, in accordance with an exemplary embodiment of the present disclosure.

FIG. 7 shows a method for conducting a psychopathology evaluation interview, in accordance with an exemplary embodiment of the present disclosure.

### DETAILED DESCRIPTION

Reference will now be made in detail to exemplary embodiments, examples of which are illustrated in the accompanying drawings. The following description refers to the accompanying drawings in which the same numbers in different drawings represent the same or similar elements unless otherwise represented. The implementations set forth in the following description of exemplary embodiments do not represent all implementations consistent with the invention. Instead, they are merely examples of apparatuses and methods consistent with aspects related to the invention as recited in the appended claims. Particular aspects of the present disclosure are described in greater detail below. The terms and definitions provided herein control, if in conflict with terms and/or definitions incorporated by reference.

Exemplary embodiments are described with reference to the accompanying drawings. Wherever convenient, the same reference numbers are used throughout the drawings to refer to the same or like parts. While examples and features of disclosed principles are described herein, modifications, adaptations, and other implementations are possible without departing from the spirit and scope of the disclosed embodiments. It is intended that the following detailed description be considered as exemplary only, with the true scope and spirit being indicated by the following claims.

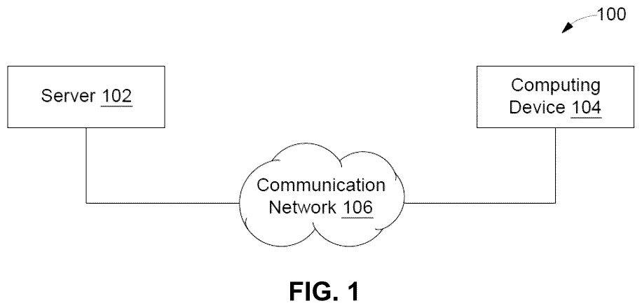

Referring now to FIG. 1, a block diagram of an environment 100 for performing psychological assessment using a generative Artificial Intelligence (AI) model is illustrated, in accordance with an exemplary embodiment of the present disclosure.

The environment 100 may include a server 102 configured for generating scripts used for performing psychological assessment using the generative AI model. It should be noted that the server 102 may be configured to generate any number of psychological assessment scripts based on user requirements. In particular, the generative AI model may be hosted on the server 102 to generate the psychological assessment scripts in response to receiving a user query. The generative AI model may automatically generate the psychological assessment scripts based on the user query. Examples of the server 102 may include, but are not limited to, an application server, a smartphone, a laptop, a desktop, a tablet, and the like. As known to a person skilled in the art, the generative AI model may be referred to as a model that is designed to answer user queries and generate new data or content (text, images, audio, and the like). The generative AI model has capabilities to answer user queries and produce new and original content that may be used in various applications, such as content generation, creative tasks, data synthesis, and the like.

The generative AI model uses unsupervised and semi-supervised machine learning algorithms to analyze a query received from the user and guides the user accordingly to resolve the query. Examples of the generative AI model include, but are not limited to, a Generative Pretrained Transformer (GPT-3, GPT-4, GPT-5, etc.), a Large Language Model (LLM), a foundation model, a Generative Adversarial Network (GAN), a variational autoencoder (VAE), a Deep Belief Network (DBN), a Recurrent Neural Network (RNN). In some embodiments, the generative AI model may be an ensembled model.

It should be noted that the generative AI model may be pretrained based on past clinical psychological assessment and treatment data. In particular, the generative AI model may be pretrained specific to the entity to administer the psychological assessment session. As will be appreciated, the generative AI model may be pretrained specifically for the entity to assist medical professionals in creating the psychological assessment scripts based on specific requirements of the entity.

Examples of the entity may include but are not limited to, a medical practitioner, a service provider, a government agency, an educational institute, a healthcare provider, a financial institute, an information technology entity, and the like. In some embodiments, the generative AI model may be pretrained based on clinic data and patient profiles (e.g., age, gender, precondition, etc.). In other words, the generative AI model may be pretrained based on variety of patient treatment data.

Further, in order to generate the psychological assessment scripts, initially, the generative AI model may receive a user query corresponding to the psychological assessment session. A user, i.e., a patient receiving the psychological assessment, may access the generative AI model via a computing device 104. The computing device 104 may be a user device. Examples of the computing device 104 may include, but are not limited to, a laptop, a desktop, a tablet, a smartphone, and the like. In other words, the user may provide the user query as an input to the generative AI model via a Graphical User Interface (GUI) of the computing device 104. In some embodiments, the generative AI model may be integrated with the computing device 104. As will be appreciated, in some exemplary embodiment, the generative AI model may correspond to a generative AI-powered live assistant bot.

Further, the user of the computing device 104 may access the generative AI model hosted on the server 102 via a communication network 106. In particular, the computing device 104 may interact with the server 102 via the communication network 106. In addition, the data used for generating the psychological assessment scripts may be stored within a database (not shown) of the server 102.

Examples of the communication network 106 may include, but are not limited to, a wireless fidelity (Wi-Fi) network, a light fidelity (Li-Fi) network, a local area network (LAN), a wide area network (WAN), a metropolitan area network (MAN), a satellite network, the Internet, a fiber optic network, a coaxial cable network, an infrared (IR) network, a radio frequency (RF) network, and a combination thereof. In an embodiment, the communication network 106 may facilitate data exchange between the computing device 104 and the server 102.

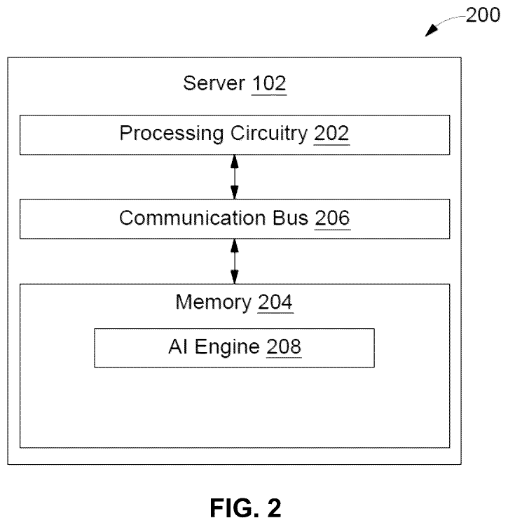

Referring now to FIG. 2, a block diagram 200 of a server 102 configured for performing psychological assessment is illustrated, in accordance with an exemplary embodiment of the present disclosure. FIG. 2 is explained in conjunction with FIG. 1. The server 102 may include a processing circuitry 202, and a memory 204 communicatively coupled to the processing circuitry 202 via a communication bus 206.

The memory 204 may store various data that may be captured, processed, and/or required by the server 102. The memory 204 may be a non-volatile memory (e.g., flash memory, Read Only Memory (ROM), Programmable ROM (PROM), Erasable PROM (EPROM), Electrically EPROM (EEPROM) memory, etc.) or a volatile memory (e.g., Dynamic Random Access Memory (DRAM), Static Random-Access memory (SRAM), etc.).

The memory 204 may also store processor-executable instructions. The processor-executable instructions, when executed by the processing circuitry 202, may cause the processing circuitry 202 to implement one or more embodiments of the present disclosure such as, but not limited to, receiving a user query. The memory 204 may include an AI engine 208. The memory 204 may also include a database (not shown) for storing data and intermediate results generated by the engine 208.

In exemplary embodiments, the AI engine 208 may generate the psychological assessment scripts. The AI engine 208 may generate the scripts in a pre-defined format. Examples of the pre-defined format may include, but are not limited to, Microsoft word (e.g., .doc, or .docx), PDF, Microsoft Visio, HTML, Markdown, Google Docs, and the like.

As will be appreciated by one skilled in the art, a variety of processes may be employed for generating the psychological assessment scripts using the generative AI model. For example, the exemplary server 102 may include the AI engine 208 configured to generate the psychological assessment scripts by the processes discussed herein. In particular, as will be appreciated by those of ordinary skill in the art, control logic and/or automated routines for performing the techniques and steps described herein may be implemented by the server 102 either by hardware, software, or combinations of hardware and software. For example, suitable code may be accessed and executed by the one or more processors on the server 102 to perform some or all of the techniques described herein. Similarly, application specific integrated circuits (ASICs) configured to perform some, or all of the processes described herein may be included in the one or more processors on the server 102.

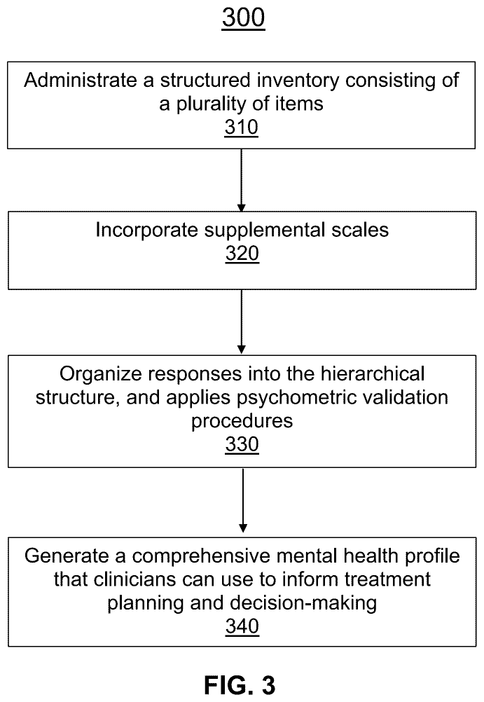

Next, FIG. 3 shows a psychological assessment method 300 designed to evaluate mental health across multiple hierarchical levels, according to some disclosed embodiments. Method 300 can be performed in the environment 100 (FIG. 1) and by server 102 (FIG. 2). For example, method 300 can be performed by the AI engine 208. Method 300 integrates Diagnostic and Statistical Manual of Mental Disorders (DSM)-based clinical syndromes with transdiagnostic dimensions and a global psychopathology factor (p-factor), enabling clinicians to generate a complete and theory-aligned profile of a patient's mental health. This approach promotes precision in psychological assessment and facilitates more effective intervention.

The method begins with the AI engine 208 administrating a structured inventory consisting of a plurality of items (step 310). For example, the inventory can include 103 items. These items are specifically designed to capture symptomatology across three hierarchically arranged levels. For example, at a first level, AI engine 208 evaluates clinical syndromes aligned with DSM categories, ensuring familiarity for clinicians. At a second level, AI engine 208 measures transdiagnostic dimensions, which include Emotional Dysregulation, Behavioral Dysregulation, and Thought Dysregulation. At a third level, AI engine 208 computes the global p-factor, representing the overall severity of psychopathology.

At step 320, AI engine 208 incorporates supplemental scales that assess cognitive complaints, body distress, eating issues, personality level, resilience, and suicidality. These scales provide clinicians with a richer understanding of the patient's mental health profile, extending beyond traditional symptom clusters.

At step 330, after data collection is complete, AI engine 208 organizes responses into the hierarchical structure, and applies psychometric validation procedures. Factor analysis confirms the replicable hierarchical structure, while internal consistency and concurrent validity are established through comparison with DSM-5 symptom measures. For example, the system has been validated in two separate adult samples (N=301 and N=640), demonstrating strong psychometric properties and clinical utility. AI engine 208 can effectively distinguish between individuals with and without a history of mental health treatment, underscoring its assessment precision.

At step 340, AI engine 208 generates a comprehensive mental health profile that clinicians can use to inform treatment planning and decision-making. Unlike legacy tools, method 300 is purpose-built to reflect contemporary dimensional models of psychopathology while maintaining compatibility with DSM conditions. Its low-burden design, strong reliability, and alignment with modern theory make it an ideal solution for the era of AI-augmented psychological assessment.

Consistent with the disclosed embodiments, method 300 offers several advantages over existing tools. It reduces clinician workload through a concise multi-item inventory, provides a multi-level assessment that captures both categorical and dimensional aspects of psychopathology, and demonstrates strong psychometric validity. Furthermore, its design anticipates integration with AI-driven clinical workflows, ensuring relevance in future mental health care systems.

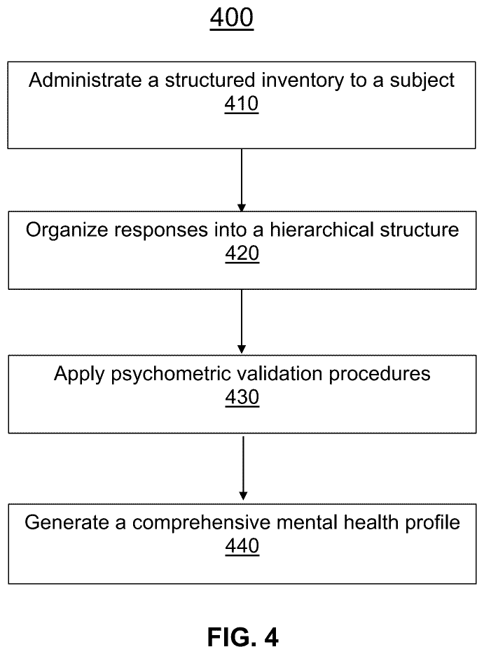

FIG. 4 shows a psychological assessment method 400 designed to evaluate psychopathology across multiple hierarchical levels of specificity, according to some disclosed embodiments. Method 400 can be performed in the environment 100 (FIG. 1) and by server 102 (FIG. 2). For example, method 400 can be performed by the AI engine 208.

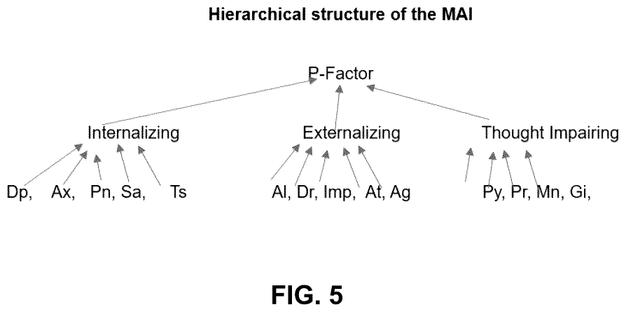

In some embodiments, the multiple hierarchical levels may include at least three levels. At the apex of the hierarchy is a global general factor, known as the p-factor, which represents overall severity of psychopathology. As shown in FIG. 5, beneath this level are three broad transdiagnostic dimensions—Internalizing, Externalizing, and Thought Impairing—that capture shared variance among related symptom clusters. At the lowest level are clinically recognizable markers, which include disorders, maladaptive traits, and symptom clusters associated with each dimension. In addition to these core scales, method 400 can incorporate supplemental constructs relevant to psychological treatment, personality structure, resilience, suicidality, and validity indicators. This multi-level approach provides clinicians with a comprehensive, theory-aligned profile that enhances psychological assessment precision and treatment planning.

As shown in FIG. 4, method 400 begins with AI engine 208 administrating a structured inventory to a subject (step 410). Items are designed to measure symptoms and traits across multiple bandwidths, enabling continuous assessment from normal to clinical ranges. Responses are recorded digitally or on paper and checked for completeness and engagement.

At step 420, after data is collected, AI engine 208 organizes responses into a hierarchical structure. For example, at a first level, AI engine 208 computes the global p-factor, which reflects general psychopathology severity. At a second level, AI engine 208 aggregates responses into three transdiagnostic dimensions: Internalizing, Externalizing, and Thought Impairing. Internalizing captures emotional distress and anxiety-related phenomena; Externalizing reflects behavioral disinhibition and substance-related behaviors; and Thought Impairing encompasses psychotic and manic symptomatology. At a third level, AI engine 208 calculates scores for clinically recognizable markers within each dimension.

In some embodiment, internalizing markers may include Depression, Anxiety, Social Anxiety, Panic, and Traumatic Stress Response. Externalizing markers include Alcohol Use, Drug Use, Impulsivity, Antisocial Behavior, and Aggression. Thought Impairing markers include Psychosis, Paranoid Ideation, Manic Activity, and Grandiose Ideation.

In addition to these core markers, AI engine 208 computes supplemental scales that provide a richer clinical picture. These supplemental scales include Eating Concerns, Bodily Distress, Personality Level, Cognitive Complaints, Suicidal Ideation, Treatment Attitude, and indicators of response style such as Too Negative and Too Virtuous. AI engine 208 also measures Well-Being, which reflects resilience and purpose, and applies validity checks using low-frequency items to ensure engagement and accurate responding.

At step 430, AI engine 208 applies psychometric validation procedures, including factor analysis and reliability checks, to confirm the hierarchical structure and internal consistency. Concurrent validity is established through comparison with DSM-based measures.

At step 440, AI engine 208 generates a comprehensive mental health profile that includes marker-level scores, mid-level dimension scores, and the global p-factor. Reports provide interpretive guidelines, note validity concerns, and suggest treatment considerations.

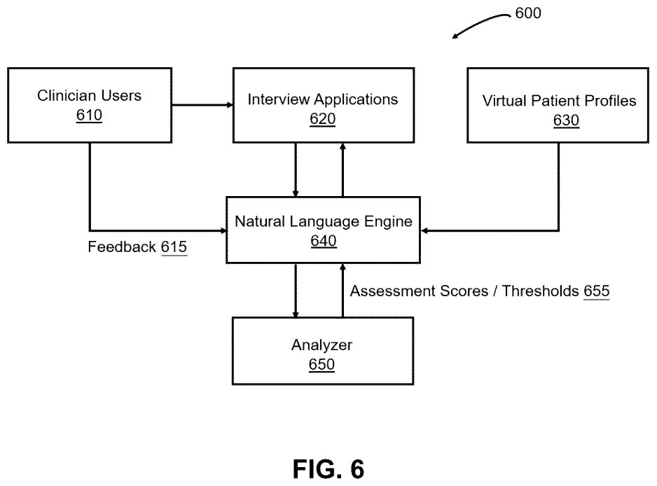

FIG. 6 is a schematic diagram showing a system 600 for conducting a psychopathology evaluation interview, according to some disclosed embodiments. As shown in FIG. 6, clinician users 610 (e.g., doctors'offices, hospitals, healthcare providers, medical practitioners, etc.) can use system 600 to conduct a psychopathology evaluation interview of a patient. During the interview, system 600 can use AI-generated scripts to generate interview questions, analyze the patient's responses, and generate a comprehensive psychopathology evaluation report about the patient. System 600 can adaptively generate the interview questions based on the patient's responses. Moreover, system 600 can analyze the patient's responses using methods 300 (FIG. 3) and 400 (FIG. 4).

As shown in FIG. 6, to conduct an interview, clinician user 610 can activate an interview application 620 installed on the clinician's computer. At the same time, a patient can activate interview application 620 installed on her device (e.g., a mobile phone, a laptop, etc.). Interview applications 620 can interact with the patient in text or voice. Interview applications 620 are in communication with a natural language engine 640, which is tasked to generate interview questions in natural language, and convert the patient's responses from natural language into machine recognizable code. To assist the generation of interview questions, natural language engine 640 can retrieve the patient's virtual patient profile 630 from a memory device. Virtual patient profile 630 may contain the patient's personal information (e.g., name, age, gender, employment history, etc.), past interview transcripts, past treatment data, etc. If the patient is a new patient without an established profile, natural language engine 640 can generate a set of initial screening questions to collect the patient's basic information to build the virtual patient profile 630. Clinician users 610 can provide feedback 615 to natural language engine 640 during or after an interview session, to adjust the interview questions based on the patient's responses. Natural language engine 640 can provide the patient's responses to analyzer 650, which in turn analyzes the responses to generate assessment scores of thresholds 655 by using, e.g., methods 300 (FIGS. 3) and 400 (FIG. 4). Assessment scores of thresholds 655 can be used by natural language engine 640 to generate additional questions.

Consistent with the disclosed embodiments, system 600 can be implemented in environment 100 (FIG. 1). For example, interview applications 620 can be installed on computing device 104. As another example, virtual patient profiles 630, natural language engine 640, and analyze 650 can be embodied in server 102.

Consistent with the disclosed embodiments, system 600 can be implemented in server 102 (FIG. 2). For example, virtual patient profiles 630 can be stored in memory 204, and natural language engine 640 and analyze 650 can be implanted by processing circuitry 202.

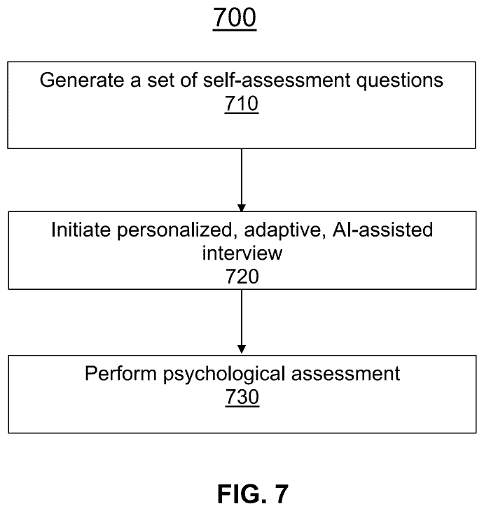

FIG. 7 shows a method 700 for conducting a psychopathology evaluation interview, according to some disclosed embodiments. Method 700 can be performed by system 600 (FIG. 6). As shown in FIG. 7, method 700 includes the following steps.

At step 710, the system generates a set of self-assessment questions for the patient, whose responses are used by the system to construct a standardized psychological inventory designed to measure symptoms and traits across multiple hierarchical levels of psychopathology. Responses to the self-assessment are scored to generate marker-level scores, transdiagnostic dimension scores, and a global general psychopathology factor.

Based on the results of this self-assessment, the system subsequently initiates a personalized, adaptive, AI-assisted interview (step 720). The interview is dynamically generated using a generative model and is conditioned on the subject's prior responses and computed scores. Interview prompts are adaptively selected according to predefined rules and learned patterns, such that elevated scores, threshold crossings, or specific response patterns trigger targeted follow-up questions relevant to those findings.

In some embodiments, the adaptive interview includes conditional branching logic, wherein the presence, severity, or configuration of specific symptom markers, transdiagnostic dimensions, or global severity indicators determines the content, sequence, and depth of subsequent questions. The interview may be used to clarify ambiguous responses, probe clinically relevant nuances, and obtain additional contextual information not fully captured by the self-assessment alone.

At step 730, the system performs psychological assessment based on the patient's responses to the interview questions. Responses obtained during the personalized interview are incorporated into the system's analytical pipeline and may refine, confirm, or contextualize the previously computed scores. This bidirectional process—wherein assessment results inform interview content, and interview responses further inform interpretation—reduces uncertainty, supports risk clarification, and mitigates false positives or false negatives arising from self-report data alone.

The system then generates a comprehensive mental health profile integrating self-assessment data and interview-derived information, including multi-level scores, interpretive guidance, and clinically relevant summaries to support evaluation and decision-making.

In some embodiments, a non-transitory computer-readable storage medium including instructions is also provided, and the instructions may be executed by a device (such as the disclosed encoder and decoder), for performing the above-described methods. Common forms of non-transitory media include, for example, a floppy disk, a flexible disk, hard disk, solid state drive, magnetic tape, or any other magnetic data storage medium, a CD-ROM, any other optical data storage medium, any physical medium with patterns of holes, a RAM, a PROM, and EPROM, a FLASH-EPROM or any other flash memory, NVRAM, a cache, a register, any other memory chip or cartridge, and networked versions of the same. The device may include one or more processors (CPUs), an input/output interface, a network interface, and/or a memory.

As used herein, unless specifically stated otherwise, the term “or” encompasses all possible combinations, except where infeasible. For example, if it is stated that a database may include A or B, then, unless specifically stated otherwise or infeasible, the database may include A, or B, or A and B. As a second example, if it is stated that a database may include A, B, or C, then, unless specifically stated otherwise or infeasible, the database may include A, or B, or C, or A and B, or A and C, or B and C, or A and B and C.

It is appreciated that the above described embodiments can be implemented by hardware, or software (program codes), or a combination of hardware and software. If implemented by software, it may be stored in the above-described computer-readable media. The software, when executed by the processor can perform the disclosed methods. The computing units and other functional units described in the present disclosure can be implemented by hardware, or software, or a combination of hardware and software. One of ordinary skill in the art will also understand that multiple ones of the above described modules/units may be combined as one module/unit, and each of the above described modules/units may be further divided into a plurality of sub-modules/sub-units.

In the foregoing specification, embodiments have been described with reference to numerous specific details that can vary from implementation to implementation. Certain adaptations and modifications of the described embodiments can be made. Other embodiments can be apparent to those skilled in the art from consideration of the specification and practice of the invention disclosed herein. It is intended that the specification and examples be considered as exemplary only, with a true scope and spirit of the invention being indicated by the following claims. It is also intended that the sequence of steps shown in figures are only for illustrative purposes and are not intended to be limited to any particular sequence of steps. As such, those skilled in the art can appreciate that these steps can be performed in a different order while implementing the same method.

In the drawings and specification, there have been disclosed exemplary embodiments. However, many variations and modifications can be made to these embodiments. Accordingly, although specific terms are employed, they are used in a generic and descriptive sense only and not for purposes of limitation.

What is claimed is:

1. A computer-implemented method for psychological assessment, the method comprising:
generating, by one or more processors, a script based on a generative model and a patient profile, wherein the generative model employs a structured psychological inventory comprising a plurality of items configured to measure symptoms and traits at multiple hierarchical levels;
receiving, by the one or more processors, responses to the script and storing the responses in a memory;
computing, by the one or more processors, a global general psychopathology factor based on the responses;
aggregating, by the one or more processors, the responses into a plurality of transdiagnostic dimensions;
calculating, by the one or more processors, a plurality of marker scores corresponding to clinically recognizable symptom clusters associated with each transdiagnostic dimension;
determining, by the one or more processors, one or more supplemental scales based on the responses; and
generating, by the one or more processors, a multi-level mental health profile for the user based on the global general psychopathology factor, the plurality of transdiagnostic dimensions, the plurality of marker scores, and the one or more supplemental scales.

2. The method according to claim 1, further comprising:
validating, by the one or more processors, a hierarchical structure and internal consistency of the computed scores using psychometric analysis.

3. The method according to claim 1, wherein the plurality of transdiagnostic dimensions comprises one or more internalizing, externalizing, or thought.

4. The method according to claim 1, wherein the markers comprise one or more of internalizing markers, externalizing markers, or thought Impairing markers.

5. The method according to claim 4, wherein the internalizing markers comprise one or more of depression, anxiety, social anxiety, panic, or traumatic stress response.

6. The method according to claim 4, wherein the externalizing markers comprise one or more of alcohol use, drug use, impulsivity, antisocial behavior, or aggression.

7. The method according to claim 4, wherein the thought impairing markers comprise one or more of psychosis, paranoid ideation, manic activity, and grandiose ideation.

8. The method according to claim 1, wherein the plurality of transdiagnostic dimensions comprises dimensions associated with internalizing, externalizing, and thought impairing.

9. An apparatus for psychological assessment, the apparatus comprising:
at least one memory for storing instructions; and
one or more processor configured to execute the instructions to cause the apparatus to perform:
generating, by one or more processors, a script based on a generative model and a patient profile, wherein the generative model employs a structured psychological inventory comprising a plurality of items configured to measure symptoms and traits at multiple hierarchical levels;
receiving, by the one or more processors, responses to the script and storing the responses in a memory;
computing, by the one or more processors, a global general psychopathology factor based on the responses;
aggregating, by the one or more processors, the responses into a plurality of transdiagnostic dimensions;
calculating, by the one or more processors, a plurality of marker scores corresponding to clinically recognizable symptom clusters associated with each transdiagnostic dimension;
determining, by the one or more processors, one or more supplemental scales based on the responses; and
generating, by the one or more processors, a multi-level mental health profile for the user based on the global general psychopathology factor, the plurality of transdiagnostic dimensions, the plurality of marker scores, and the one or more supplemental scales.

10. A non-transitory computer readable storage medium storing a set of instructions that are executable by one or more processing devices to cause an apparatus to perform a method comprising:
generating, by one or more processors, a script based on a generative model and a patient profile, wherein the generative model employs a structured psychological inventory comprising a plurality of items configured to measure symptoms and traits at multiple hierarchical levels;
receiving, by the one or more processors, responses to the script and storing the responses in a memory;
computing, by the one or more processors, a global general psychopathology factor based on the responses;
aggregating, by the one or more processors, the responses into a plurality of transdiagnostic dimensions;
calculating, by the one or more processors, a plurality of marker scores corresponding to clinically recognizable symptom clusters associated with each transdiagnostic dimension;
determining, by the one or more processors, one or more supplemental scales based on the responses; and
generating, by the one or more processors, a multi-level mental health profile for the user based on the global general psychopathology factor, the plurality of transdiagnostic dimensions, the plurality of marker scores, and the one or more supplemental scales.
